(19) United States
(12) Patent Application Publication
(10) Pub. No.: US 2011/0040355 A1
(43) Pub. Date: Feb. 17, 2011

(54) Phototherapy mask

(76) Inventor: Stacy Francis, Oak Ridge, NJ (US)

Correspondence Address: THOMAS J. GERMINARIO, ESQ., 154 ROUTE 206, CHESTER, NJ, 07930, US

(21) Appl. No.: 12/461,438

(22) Filed: Aug. 12, 2009

(51) Int. Cl. A61N 5/06

(52) U.S. Cl. 607/88

(57) ABSTRACT

A phototherapy mask uses optical fibers coupled to LEDs to irradiate a treated epidermal skin area on or around a person's face with specific wavelengths of light in selected dosages (J/cm²). Peripheral configuration of LEDs on the mask eliminates problems of heat dissipation, and multi-mode optical fiber is employed for diffusion of light uniformly over the treated epidermal skin area.

### BACKGROUND OF THE INVENTION

The present invention relates to therapeutic devices, and more specifically to devices for administering external light therapy, also known as phototherapy, to organic tissue, and particularly to the epidermal layer of the human skin, in order to treat various medical conditions.

Certain portions of the infrared, visible, and ultraviolet light spectra have proven to provide efficacious treatment for several organic disorders, including, for example, infantile jaundice and seasonal affective disorder. In particular, various skin conditions respond favorably to certain light spectra, including acne, lesions, and broken capillaries. Red/infrared light treatment has been used to reverse epidermal damage and induce production of collagen and elastin in order to restore a more youthful appearance to the skin. Blue/violet light therapy is an efficacious treatment for acne and skin lesions.

Epidermal phototherapy treatments are currently administered by licensed aestheticians using probes and/or mats comprising an array of colored light-emitting diodes (LEDs). This mode of treatment has several disadvantages, however. In treating a large irregularly-shaped area, such as the face, it's not practical to use a flat mat, and instead the aesthetician must move an LED probe around step-wise in a grid pattern to treat the entire skin area. In facial phototherapy treatments, this is typically done by starting at the forehead and moving the probe to a different placement location every 30-60 seconds. Since a standard phototherapy probe covers 5 square centimeters (cm²) and the average surface area of an adult human face is 370 cm², the probe must be moved at least 74 times in order to treat the entire face. At 30-second placement intervals, this LED probe facial treatment process will therefore take 37 minutes, while at 60-second intervals it will take 74 minutes. Since the aesthetician is fully occupied during this entire process, the lengthy duration of LED probe facial treatments greatly adds to their expense.

The basic concept of the present invention is to provide a mask for facial phototherapy treatments that will enable the entire skin area of the face and adjoining skin areas, such as the neck and decollete (collectively referred to hereinafter as the “face” or “facial areas”) to be treated simultaneously, rather than in the step-wise fashion required by LED probes. Using such a mask, the entire facial skin area can receive a dosage of light energy, measured in Joules per square centimeter (J/cm²), equivalent to that of the LED probe process in a fraction of the time. Moreover, once the mask is placed on the patient, the aesthetician is free to perform other tasks, thus reducing treatment expenses.

The approach of the prior art to the design of a phototherapy facial mask has been to use multiple LEDs distributed over the inner surface of the mask to illuminate the skin directly. The “Therapeutic Facial Mask” patent (U.S. Pat. No. 5,913,883) issued to Alexander et al., discloses a rudimentary face mask lined with LEDs for therapeutic light treatments. This design is quite problematic, however, because it does not address LED heat build-up within the confined mask interior, nor does it provide a means of diffusing the LED light to achieve uniform light distribution over the treated facial areas. Moreover, because the LEDs are not in contact with the skin, there is substantial reflective light loss off the surface of the skin. FIG. 3 shows the LEDs to be embedded within the inner surface of the mask, such that the wiring will be expensive to fabricate and repair.

More sophisticated, but nonetheless problematic, is the phototherapy system described in “Flexible Illuminators for Phototherapy” (U.S. Pat. No. 6,290,713) issued to Russell in 2001. This invention is primarily directed toward a flexible mat or pad, in which LEDs and associated wiring are embedded in a flexible substrate with a light reflecting back surface. While means for dissipating heat and diffusing light within the flexible substrate are proposed, they are vague, difficult to implement and of uncertain effectiveness. A network of interface channels surrounding the LEDs is proposed, with an unspecified fluid cooling medium to be pumped through the channels. Introduction of refractive materials within the interface channels is broadly suggested as a means of diffusing the LED light, but there is no explanation as to how this would achieve a uniform light energy dosage to all skin areas. Wiring within the substrate is intricate and expensive, and such wiring does not appear practical for a complex curvilinear configuration such as a facial mask—although a mask embodiment is illustrated in FIG. 5A.

In FIG. 1 of the Russell patent is depicted a “prior art” design, in which the illuminator is a fiber-optic mat. The Russell patent specification (column 2, lines 44-66) states that such fiber-optic illuminators are less efficient than LED versions because of the need for a high-intensity light source, such as a halogen lamp. But technological advances since the issuance of the Russell patent now enable the use of efficient LEDs as light sources for fiber-optic phototherapy mats, as taught by the Williams patent (U.S. Pat. No. 7,479,664).

The new LED technology makes it feasible to fabricate a phototherapy mask out of woven or unwoven strands of optical fibers, with LED light sources arranged in the periphery of the mask. This design of the present invention reduces the problems of heat dissipation and light diffusion to a manageable level. Since optical fibers can have direct skin contact, moreover, the loss of light by reflection is greatly reduced. Circuitry and wiring is also greatly simplified, and an optical fiber mask can conform to facial contours much more readily than a LED-embedded flexible substrate. Optical fibers can also be configured in multiple layers, thus affording more variety of therapeutic options.

### SUMMARY OF THE INVENTION

The effective use of optical fibers to create a phototherapy mask is not a simple matter, but requires that several technical problems first be solved. Not all types of optical fibers are suitable for this use. Optical fibers developed for communications uses, for example, are not capable of being effectively coupled to an LED light source and will not allow light energy to be uniformly emitted along the length of the fiber. In order to address the source-coupling and light-diffusion problems, it's necessary to understand how optical fibers transmit light.

Referring to FIG. 1, an optical fiber consists of two layers—an inner core and outer cladding. Light entering the core is confined there by a phenomenon known a “total internal reflection”. Total internal reflection occurs when a light ray encounters a boundary between a medium having a relatively high index of refraction and a medium having a relatively low index of refraction. The more oblique the incidence angle of the light ray to the boundary, the less light will cross the boundary and the more will be reflected back. When the incidence angle of the light ray becomes sufficiently oblique, no light will cross the boundary, and there will be total internal reflection. A familiar example of this phenomenon occurs when light passes from water into air, with water having a higher index of refraction than air. When the incidence angle of the light ray, measured from the perpendicular to the water surface, exceeds about 48°, the light will be totally reflected at the surface. This explains the mirror-like appearance of a water surface when viewed from below.

Returning to the optical fiber of FIG. 1, the difference between the index of refraction of the core and the cladding will determine the range of incidence angles of light entering the core that will be confined there by total internal reflection. This range of incidence angles defines an “acceptance cone” through which the source light must be focused in order to effectively couple with the fiber. The task of determining the extent of the acceptance cone involves calculation of a “numerical aperture”, abbreviated as NA, which is illustrated in FIG. 2.

FIG. 2 depicts a section of optical fiber showing the boundaries between the core and cladding as solid lines, the longitudinal axis of the core as a dark dashed line, and the perpendicular to the core/cladding boundary as a light dashed line. The index of refraction of the core is designated as n₁, while the refractive index of the cladding is n₂. The numerical aperture NA is the sine of the maximum incidence angle α that a light ray can have to the longitudinal axis of the core in order to be totally confined within the core. The formula for numerical aperture is given as:

NA=sine α=√(n₁²−n₂²)

The “acceptance angle”, which defines the cone of acceptance, is twice the angle α. Within the core, a totally confined light ray must exceed the “critical angle” θ_{c} with respect to the perpendicular to the core/cladding boundary, as shown in FIG. 2. The sine of the critical angle is determined by the ratio of the index of refraction of the cladding to that of the core, in accordance with the formula:

θ_{c}=arcsin(n₂/n₁)

To create an LED-driven optical fiber mask, it's essential that the optical fibers be effectively coupled with an LED, such that there's not excessive escape of light energy from the core of the fiber. To achieve the requisite coupling, the acceptance angle 2α of the fiber should be about 60°, which equates to a numerical aperture NA of 0.5. Therefore, referring to the formula for NA given above, the core and cladding materials of the optical fiber must be specified so that difference of the squares of their respective indices of refraction is approximately 0.25. Certain types of plastic optical fiber (POF) can meet these specifications. For example, optical fiber having a core made of polymethyl methacrylate (PMMA), with an index of refraction n₁ of 1.49, and cladding made of a fluorinated polymer, with an index of refraction of n₂ of 1.40, would achieve efficient LED coupling. Applying the formulas given above, the acceptance angle 2α for such a fiber would be 61° and the critical angle θ_{c} would be 70°. POF fiber is suitable for low-speed, short-distance applications, such as the present invention, and has the advantages of being much less expensive and much more flexible than glass optical fiber.

The second problem that must be addressed in designing an LED-driven optical fiber phototherapy mask is uniform distribution of light energy over the entire facial area. Again, fiber specifications must be selected so as to maximize the diffusion and distribution of light as it travels along the core. This is opposite to the objective in selecting optical fiber for telecommunications and digital applications, in which discrete data pulses are transmitted and diffusion causes loss of data. The preferred optical fiber for data applications, therefore, is “single-mode fiber”. Single-mode fiber support only one mode of light propagation through the core, which mode follows the longitudinal axis of the core, as depicted in FIG. 3c. Since there is only one light path through the fiber, a data pulse entering one of the fiber will not “spread out” or diffuse as it travels through the fiber, thereby keeping the data pulse intact. Single-mode fiber must have a very low numerical aperture and a very small core diameter. This means that its acceptance angle and acceptance cone will be very narrow-too narrow to allow efficient coupling with an LED light source.

As illustrated in FIGS. 3a and 3b, “multi-mode fiber” supports more than one mode of light propagation through the core. In multi-mode fiber, light propagates down the core along multiple paths, some close to the longitudinal axis and others at various angles greater than the critical angle θ_{c}. Since light rays at different angles will have different path lengths, they will take different times to traverse the fiber. This causes the light energy in a multi-mode fiber to spread out and diffuse, tending to distribute the light energy uniformly along the length of the fiber. Such diffusion in multi-mode fibers can be reduced by using a “graded index” core/cladding boundary, in which the decrease of refractive index is gradual, as shown in FIG. 3b. On the other hand, diffusion can be maximized by using a “step index” boundary, in which the decrease of refractive index is abrupt, as shown in FIG. 3a.

Consequently, to achieve optimal uniformity of light energy dosage in an LED-driven optical fiber phototherapy mask, a step-index multi-mode fiber must be used. This choice also is consistent with LED-coupling imperatives discussed above, since step-index multi-mode fibers have large core diameters and high numerical apertures, so that they are very efficient at collecting light. Step-index multi-mode fiber is also capable of transmitting more power than other fiber types, thus allowing fewer fibers to be used to meet the phototherapy dosage requirements.

Dosage uniformity along the fiber depends not only on uniform distribution of light energy within the fiber, but also on uniform light emission from the fiber. The reflection of emitted light from the skin surface must also be considered. In order for light to escape from the core of the fiber, there must be provided gaps in the cladding. In the Williams patent (U.S. Pat. No. 7,479,664), the suggested method of providing such gaps in the cladding is simply to remove the cladding by mechanical or chemical means (col. 3, ln. 52-57), in effect creating holes in the cladding. But such holes will be filled with air, skin moisture, or a combination of both. Since both air (n=1) and water (n=1.33) have indices of refraction that are less than that of the cladding, a large percentage of the light rays will be reflected from the core/air and/or core/water boundary at the hole and never reach the skin. Many of the rays that do pass through the holes in the cladding will strike the skin at a very oblique angle and be lost by reflection. So the Williams design lacks an efficient means of getting the light energy from the core of the fiber into the skin.

In the present invention gaps are provided in the underside of the fiber cladding, and the gaps are filled with a material having a refractive index that is equal to or, preferably, greater than that of the core. Using a fill material in the gaps with a higher refractive index than the core has the dual effects of increasing the percentage of light transmitted through the core/gap boundary and bending the transmitted light toward the boundary perpendicular. The latter effect means that the transmitted light will strike the skin surface at a less oblique angle, thereby reducing reflective losses. Optimally, an inexpensive transparent plastic material having an index of refraction greater than that of the core material is selected to fill the cladding gaps. An example of suitable gap-filling material is polyethylene (n=1.51). Used in conjunction the exemplary plastic optical fiber described above, with a core of PMMA (n=1.49), the polyethylene gap would bend a light ray incident at the critical angle θ_{c} of 70° toward the boundary perpendicular to an emission angle of θₑ in accordance with Snell's Law, as illustrated in FIG. 4:

sin θ₂/sin θ₁=n₁/n₂ sin θₑ=(1.49/1.51)sin 70°=0.927 θₑ=68°

Even more efficient gap-filling material in terms of limiting reflective light losses are the polymers described in U.S. Pat. No. 5,422,422 of Bader et al. (n=1.60). Again applying Snell's Law:

sin θₑ=(1.49/1.60)sin 70°=0.875 θₑ=61°

Optimally, the width of the emission gaps would be in the range of 1-3 mm, in order to allow light rays reflected off the skin surface to re-enter the core and not be lost. In order to recover as much skin-reflected light as possible, the exterior surface of the mask can be lined with a reflective material, such as metalized Mylar.

Since light emissions through the gaps will progressively attenuate in light energy density in the fiber as it extends further from the LED source, a compensatory feature is needed in order to achieve dosage uniformity across the mask. One potential compensatory feature is to increase the frequency and/or width of the emission gaps further from the LED source. Alternately, the fibers comprising the mask could be looped so that both ends are coupled to the LED source. Yet another alternative is to have each end of the fiber coupled to a different LED source. Still another compensatory feature is to interlace the optical fibers comprising the mask so that alternating adjoining fibers are lit from opposite ends. Combinations of one or more of the foregoing compensatory features can also be applied.

The third problem that must be addressed in designing an LED-driven optical fiber phototherapy mask is that of heat dissipation. Since the present invention does not require that the LEDs be placed in close proximity to the treated skin areas, as does the prior art, this problem becomes considerably more tractable. In the present invention, LEDs are positioned around the perimeter of the mask and are connected to heat sinks with heat dissipating means, such as fins, extending outward from the mask perimeter. Therefore, sufficient cooling is provided by the ambient atmosphere, with no potential for heat buildup with the mask, as in the patent of Alexander, et al., and without the needs for complex pumping of fluid coolants through the device, as in the Russell patent.

In the present invention, light from the LEDs is focused by one or more lenses toward bundled ends of multiple optical fibers within the applicable acceptance angle of the fiber. Optionally, a filter may be placed between the LED and the lens to select the appropriate wavelength of light for the desired phototherapy treatment. If a filter is not used, the colors of the LEDs themselves are selected for the desired treatment wavelengths.

The number and wattage of the LEDs can be determined from the light energy dosage and treatment time. Consider, for example, a 6 J/cm² dosage to be applied by the mask to the entire surface of a face during a 5-minute treatment. Taking the surface area of the face at 370 cm², the total dosage is 2220 Joules. Distributed over 5 minutes, this dosage requires a total power output of 7.4 watts. Conservatively allowing for 30% light energy loss by reflection and fiber transmission attenuation, the total power requirement is 12 watts. This requirement is supplied by 12 one-watt LEDs, which are distributed around the mask perimeter.

Other features, objects and advantages of the present invention will become apparent from the following descriptions, taken in connection with the accompanying drawings, in which, by way of illustration and example, a preferred embodiment of the present invention is disclosed.

### BRIEF DESCRIPTION OF THE DRAWINGS

FIG. 1 is a cross-section view of optical fiber showing the core, cladding and acceptance cone.

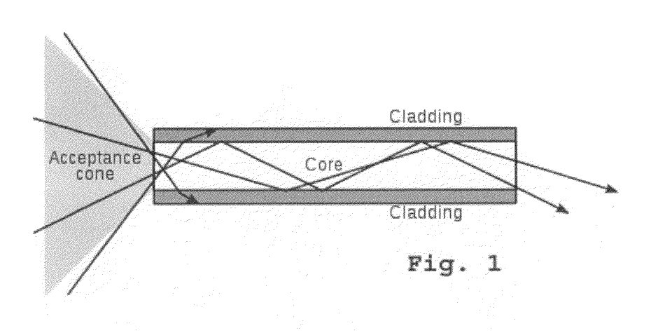

FIG. 2 is a schematic depiction of fiber-optic numerical aperture, acceptance angle, and critical angle.

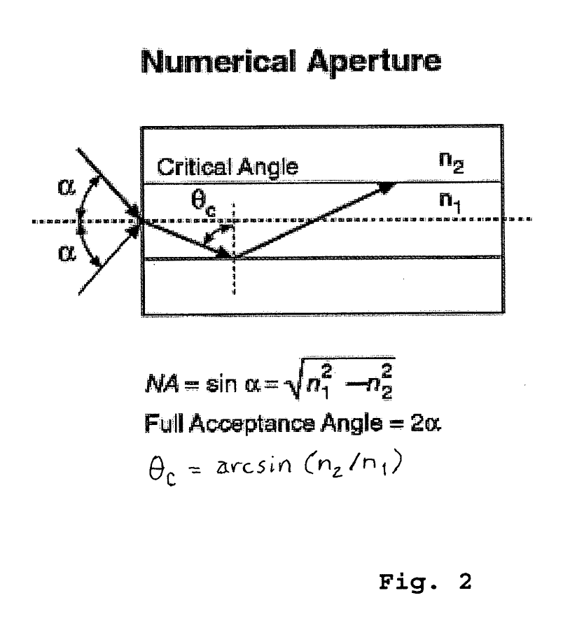

FIGS. 3a, 3b, and 3c depict the refractive indices and transmission modes of multi-mode step-index, multi-mode graded-index, and single-mode fiber, respectively.

FIG. 4 is a schematic illustration of Snell's Law.

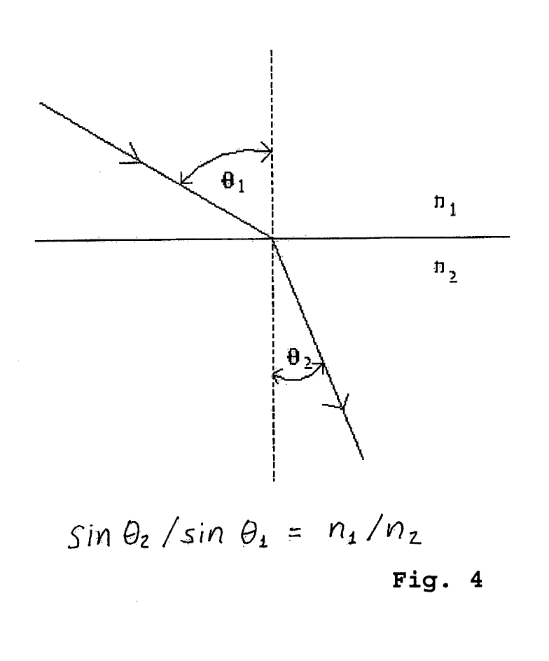

FIG. 5 is front view, with the outer Mylar reflective layer cut away, of a phototherapy mask according to the preferred embodiment of the present invention.

FIG. 6 is a cross-section detail of one of the light modules of a phototherapy mask according to the preferred embodiment of the present invention.

### DETAILED DESCRIPTION OF THE PREFERRED EMBODIMENT

The following detailed description of the preferred embodiment is meant to be illustrative of one specific design of the present invention. The number and arrangement of the components described herein is not intended to limit the scope of the invention. The number and arrangement of components can be altered to conform to various treatment and dosage requirements. Therefore, the detailed design presented herein is presented for exemplary purposes only.

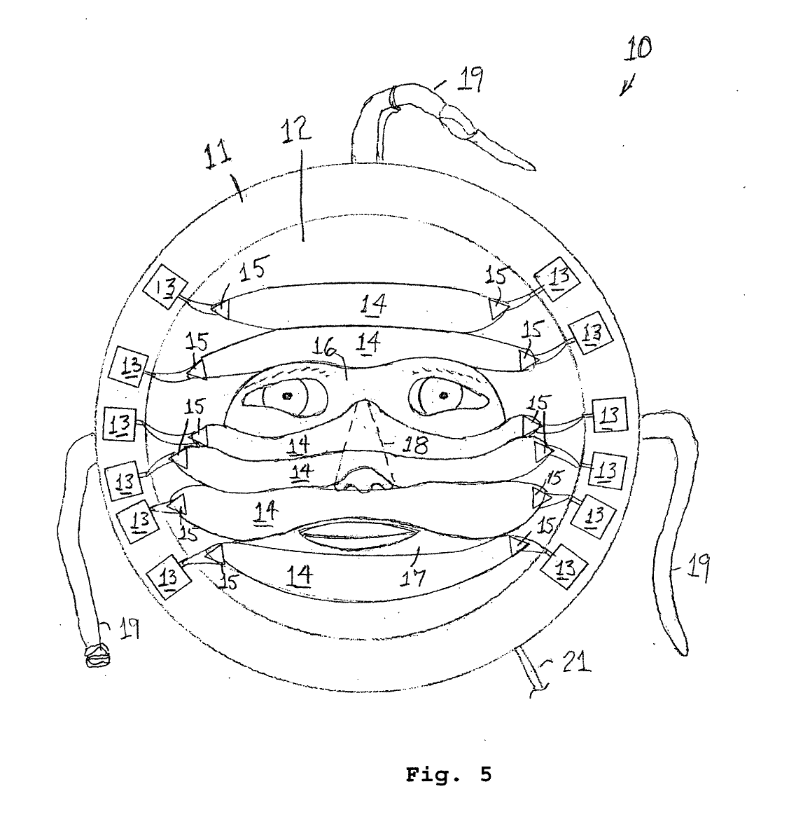

Referring to FIG. 5, the phototherapy mask representing the preferred embodiment of the present invention 10 comprises a perimeter 11, which is an annular sheet of elastically deformable plastic material that is attached to the circumference of a hub 12, which is a circular, elliptical or oval sheet of flexible, transparent plastic having a refractive index greater than 1.5. Attached to the perimeter 11 are twelve light modules 13, each of which is coupled to one end of one of six fiber-optic bundles 14. Each fiber-optic bundle 14 comprises 200 optical fibers, each approximately 1 mm in diameter and 20 inches in length, crimped together at either end by a ferrule type connector 15, which is anchored to the hub 12. By coupling the fiber-optic bundles 14 at both ends to a light module 13, the attenuation in light energy density in the fiber 14 as it extends further from the light source 13 is compensated, ensuring a uniform energy dosage across the mask, as explained above.

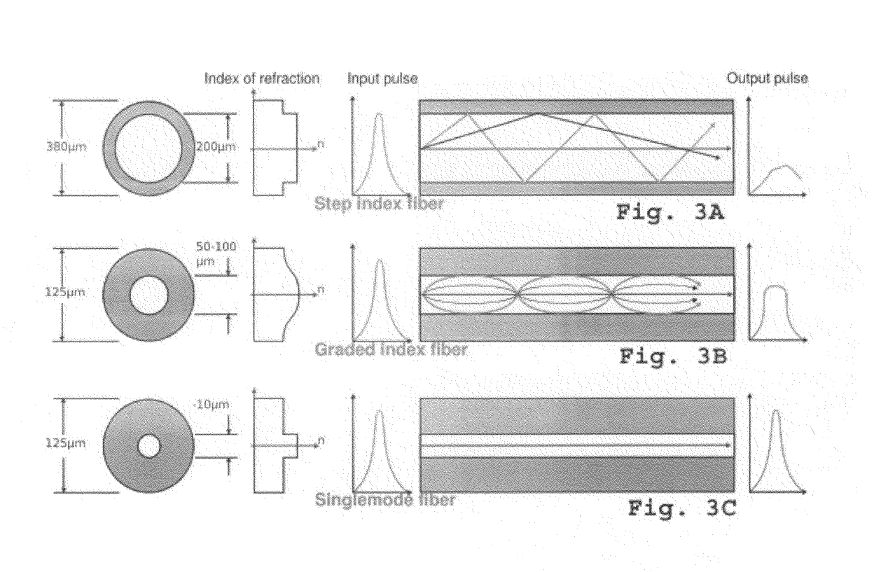

The optical fibers are preferably plastic, with a core of polymethyl methacrylate (PMMA) and a cladding of fluorinated polymer, such that the fibers have a numerical aperture (NA) of approximately 0.5 and an acceptance angle of approximately 60°. The optical fibers are of the multi-mode, step-index type illustrated in FIG. 3a. Between the ferrules, the individual optical fibers comprising the fiber optic bundles 14 are spread out and intertwined so as to form a flat plait, the underside of which is plastically fused to the material of the hub 12. On the underside of each optical fiber in the bundle 14, there are a series of 1 mm gaps in the cladding at intervals of 20 mm, such that the plastic material of the hub 12 fills the gaps. By using the higher refractive index hub material 12 to fill the gaps, reflective losses at the core/gap boundary and from the skin surface are minimized, as explained above.

The hub 12 has an eye slot 16, a mouth slot 17, and nose bridge, and it is generally contoured to fit over a human face. The mask 10 is snuggly fitted to the face by a series of adjustable straps. Preferably, a removable layer of reflective Mylar (not shown) forms the outer covering of the mask 10.

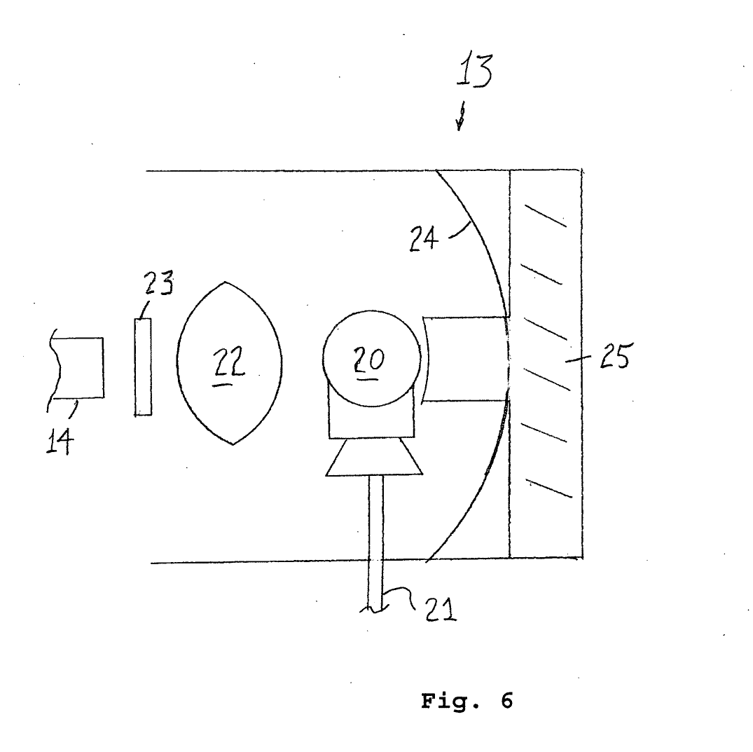

Referring to FIG. 6, the light modules 13 comprise an LED 20, a power cable 21, a focusing lens 22, color filters 23, a reflector 24, and a heat sink 25. The LED 20 is one-watt, white light, surface-mountable, high light density, preferably rated at least 100 lumens per watt @ 350 mA. A transformer (not shown) supplies 3.2 V, 350 mA DC electrical power to the LED through the power cable 21. The light from the LED 20 is focused on the end of the fiber-optic bundle 14, within its cone of acceptance, by the focusing lens 22. Interchangeable color filters 23 between the focusing lens 22 and the fiber-optic bundle 14 are used to select the proper wavelength of light for the desired phototherapy treatment. A spherical or parabolic reflector 24 in back of the LED 20 reflects stray light back toward the focusing lens 22. The heat sink 25 dissipates heat from the LED 20 to the ambient air.

Although a preferred embodiment of the invention has been disclosed for illustrative purposes, those skilled in the art will appreciate that many additions, modifications and substitutions are possible, without departing from the scope and spirit of the present invention as defined by the accompanying claims.

What is claimed is:

1. A phototherapy mask for applying light energy in a selected dosage (J/cm²) to a treated epidermal skin area on or around a person's face, the phototherapy mask comprising:
(a) multiple light modules, each light module comprising one or more LED(s) which emit light and one or more electrical connections by which electrical power is supplied to the LED(s);
(b) multiple fiber-optic bundles, each fiber-optic bundle comprising multiple optical fibers, and each optical fiber comprising a central core and an outer cladding, wherein the index of refraction of the core exceeds the index of refraction of the cladding, and wherein each fiber-optic bundle is coupled to one or more LED(s), such that the optical fibers comprising the fiber-optic bundle receive and transmit the light emitted from the LED(s) through the core along the length of the optical fibers, and wherein the cladding of each optical fiber has gaps at intervals along the length of the optical fiber, which gaps permit portions of the light transmitted through the optical fiber to leave the core and irradiate the treated epidermal skin area; and
(c) a mask structure, contoured to fit over the face and having means of attachment to the face, to which mask structure the light modules and the fiber-optic bundles are secured.

2. The phototherapy mask according to claim 1, wherein each of the optical fibers has an upper side and an underside, such that the underside is oriented toward the epidermal skin areas, and wherein the gaps in the cladding of each optical fiber are on the underside of the optical fiber.

3. The phototherapy mask according to claim 2, wherein the gaps in the cladding of each optical fiber are filled with a flexible transparent material having an index of refraction equal to or greater than the index of refraction of the core of the optical fiber.

4. The phototherapy mask according to claim 3, wherein each of the light modules further comprises one or more focusing lenses, which focusing lens(es) focus (es) the light emitted by the LED(s) within the acceptance angle of the optical fibers, such that all of the light is transmissively confined within the core of the optical fibers.

5. The phototherapy mask according to claim 4, wherein the optical fibers are of the multi-mode type.

6. The phototherapy mask according to claim 5, wherein the optical fibers are of the step-index type.

7. The phototherapy mask according to claim 6, wherein each fiber optic bundle has two bundle ends, which bundle ends are coupled to separate LEDs, such that the treated epidermal skin area is uniformly irradiated in accordance with the selected dosage.

8. The phototherapy mask according to claim 7, wherein the core and cladding of each of the optical fibers consist of flexible, transparent plastic.

9. The phototherapy mask according to claim 8, wherein the diameter of each of the optical fibers is approximately 1 mm.

10. The phototherapy mask according to claim 9, wherein the core of each of the optical fibers consists of polymethyl methacrylate (PMMA), and wherein the cladding of each of the optical fibers consists of a fluorinated polymer.

11. The phototherapy mask according to claim 10, wherein each of the light modules further comprises a heat sink, comprising a material having a high coefficient of thermal conductivity, such that the heat sink conducts heat generated by the LED(s) away from the light module and into ambient air surrounding the mask.

12. The phototherapy mask according to claim 1, wherein each of the light modules further comprises multiple interchangeable color filters, by means of which specific wavelengths of the light emitted by the LED(s) are selected for transmission to the optical fibers.

13. The phototherapy mask according to claim 12, further comprising a reflective covering sheet that is removably attachable over the mask structure, which reflective covering sheet reflects light from the fiber-optic bundles back toward the treated epidermal skin area.
